(19) United States
(12) Patent Application Publication
(10) Pub. No.: US 2020/0265957 A1
(43) Pub. Date: Aug. 20, 2020

(54) METHOD FOR OPERATING AN ELECTRONIC DEVICE, APPARATUS FOR WEIGHT MANAGEMENT BENEFIT PREDICTION, AND STORAGE MEDIUM

(71) Applicant: BOE TECHNOLOGY GROUP CO., LTD., Beijing (CN)

(72) Inventors: Hui Du, Beijing (CN); Shuai Cao, Beijing (CN); Jinye Liu, Beijing (CN)

(21) Appl. No.: 16/563,755

(22) Filed: Sep. 6, 2019

(30) Foreign Application Priority Data
Feb. 15, 2019 (CN) 201910117019.7

(51) Int. Cl. G16H 50/30; G16H 50/70; A61B 5/00

(52) U.S. Cl. CPC G16H 50/30; A61B 5/4869; G16H 50/70

(57) ABSTRACT

A method for operating an electronic device includes: determining a plurality of influence parameters associated with a target disease, the influence parameters at least include a body mass index; determining calculation parameters according to the influence parameters, and determining a risk prediction model for the target disease based on the calculation parameters; collecting medical diagnosis information of an object to be tested corresponding to the influence parameters, and inputting the medical diagnosis information to the risk prediction model to obtain a first risk prediction value; substituting a value of the body mass index in the medical diagnosis information with a weight management target value, inputting the medical diagnosis information to the risk prediction model again to obtain a second risk prediction value; and calculating a weight management benefit prediction value corresponding to the weight management target value according to the first risk prediction value and the second risk prediction value.

### CROSS REFERENCE

The present application claims priority to Chinese Patent Application No. 201910117019.7 and filed Feb. 15, 2019, the entire contents of which are incorporated herein by reference.

### TECHNICAL FIELD

The present disclosure relates to the field of computer technologies, and more particularly, to a method for operating an electronic device, an apparatus for weight management benefit prediction, and a computer-readable storage medium.

### BACKGROUND

According to a survey report published in the famous British medical journal The Lancet in 2016, it was found that China has become a country with the most obesity population in the world. In addition, according to data from the National Bureau of Statistics and the National Health and Family Planning Commission, the Chinese people's overweight and obesity rates are rising. From 1992 to 2015, the overweight rate rose from 13% to 30%, and the obesity rate rose from 3% to 12%. Controlling overweight and obesity through personalized, scientific and reasonable weight management has become one of important tasks of disease control in China. However, due to the lack of quantitative prediction methods for weight management benefits currently, it is challenging to intuitively reflect the significance of weight management for disease prevention, and thus it is difficult to mobilize the enthusiasm of overweight, obese or high-risk groups to manage weight.

It is to be noted that the above information disclosed in this Background section is only for enhancement of understanding of the background of the present disclosure and therefore it may contain information that does not form the related art that is already known to a person of ordinary skill in the art.

### SUMMARY

An objective of the present disclosure is to provide a method for operating an electronic device, an apparatus for weight management benefit prediction, and a computer-readable storage medium.

According to an aspect of the present disclosure, there is provided a method for operating an electronic device. The method includes determining a plurality of influence parameters associated with a target disease. The influence parameters at least include a body mass index. The method includes determining a plurality of calculation parameters according to the plurality of influence parameters, and determining a risk prediction model for the target disease based on the plurality of calculation parameters. The method includes collecting medical diagnosis information of an object to be tested corresponding to the plurality of influence parameters, and inputting the medical diagnosis information to the risk prediction model to obtain a first risk prediction value. The method includes substituting a value of the body mass index in the medical diagnosis information with a weight management target value, and inputting the medical diagnosis information to the risk prediction model again to obtain a second risk prediction value. The method includes calculating according to the first risk prediction value and the second risk prediction value to obtain a weight management benefit prediction value corresponding to the weight management target value.

In an example arrangement of the present disclosure, the risk prediction model is Z=1−a\hat{ }(sum(βᵢ*Xᵢ)−b). Z represents a risk prediction value of the target disease, Xᵢ represents the plurality of calculation parameters, βᵢ represents a preset weight coefficient of the plurality of calculation parameters, and a and b represent preset regulation coefficients.

In an example arrangement of the present disclosure, the risk prediction model is Z=1−c\hat{ }(sum(βi*(Yi−Xi))) Z represents a risk prediction value of the target disease, Xᵢ represents the plurality of calculation parameters, βᵢ represents a preset weight coefficient of the plurality of calculation parameters, c represents a preset regulation coefficient, and Yᵢ represents preset reference values of the plurality of calculation parameters.

In an example arrangement of the present disclosure, the influence parameters include numerical parameters and non-numerical parameters.

Before determining a plurality of calculation parameters according to the plurality of influence parameters, the method further includes converting the non-numerical parameters in the influence parameters into the numerical parameters.

In an example arrangement of the present disclosure, the calculation parameters include original parameters, first-order parameters, and second-order parameters.

Determining a plurality of calculation parameters according to the plurality of influence parameters includes: determining a part of parameters among the plurality of influence parameters as the original parameters; calculating a part of parameters among the plurality of influence parameters according to a first preset formula to obtain the first-order parameters; and calculating a part of parameters and another part of parameters among the plurality of influence parameters according to a second preset formula to obtain the second-order parameters.

In an example arrangement of the present disclosure, inputting the medical diagnosis information to the risk prediction model to obtain a first risk prediction value includes: determining, according to the medical diagnosis information, whether the object to be tested satisfies an assessment condition; and inputting the medical diagnosis information to the risk prediction model when determining that the object to be tested satisfies the assessment condition to obtain the first risk prediction value.

In an example arrangement of the present disclosure, the assessment condition includes the body mass index of the object to be tested being greater than a preset threshold.

In an example arrangement of the present disclosure, determining a plurality of influence parameters associated with a target disease includes querying a preset mapping relationship table to determine the plurality of influence parameters associated with the target disease. The mapping relationship table is used for providing a mapping relationship between various diseases and the influence parameters.

According to another aspect of the present disclosure, there is provided an apparatus for weight management benefit prediction. The apparatus includes a parameter determining module configured to determine a plurality of influence parameters associated with a target disease. The influence parameters at least include a body mass index. The apparatus includes a model determining module configured to determine a plurality of calculation parameters according to the plurality of influence parameters, and determine a risk prediction model for the target disease based on the plurality of calculation parameters. The apparatus includes a first prediction module configured to collect medical diagnosis information of an object to be tested corresponding to the plurality of influence parameters, and input the medical diagnosis information to the risk prediction model to obtain a first risk prediction value. The apparatus includes a second prediction module configured to substitute a value of the body mass index in the medical diagnosis information with a weight management target value, and input the medical diagnosis information to the risk prediction model again to obtain a second risk prediction value. The apparatus includes a benefit prediction module configured to calculate according to the first risk prediction value and the second risk prediction value to obtain a weight management benefit prediction value corresponding to the weight management target value.

According to still another aspect of the present disclosure, there is provided a computer-readable storage medium, which stores a computer program. The computer program is executable by a processor to: determine a plurality of influence parameters associated with a target disease, the influence parameters at least including a body mass index; determine a plurality of calculation parameters according to the plurality of influence parameters, and determine a risk prediction model for the target disease based on the plurality of calculation parameters; collect medical diagnosis information of an object to be tested corresponding to the plurality of influence parameters, and input the medical diagnosis information to the risk prediction model to obtain a first risk prediction value; substitute a value of the body mass index in the medical diagnosis information with a weight management target value, and input the medical diagnosis information to the risk prediction model again to obtain a second risk prediction value; and calculate according to the first risk prediction value and the second risk prediction value to obtain a weight management benefit prediction value corresponding to the weight management target value.

It is to be understood that the above general description and the detailed description below are merely example and explanatory, and do not limit the present disclosure.

### BRIEF DESCRIPTION OF THE DRAWINGS

The accompanying drawings herein are incorporated in and constitute a part of this specification, illustrate arrangements conforming to the present disclosure and, together with the description, serve to explain the principles of the present disclosure. Apparently, the accompanying drawings in the following description show merely some arrangements of the present disclosure, and persons of ordinary skill in the art may still derive other drawings from these accompanying drawings without creative efforts.

FIG. 1 schematically illustrates a flowchart of blocks of executing a method for weight management benefit prediction by an electronic device according to an example arrangement of the present disclosure;

FIG. 2 schematically illustrates a flowchart of a part of blocks of executing a method for weight management benefit prediction by an electronic device according to another example arrangement of the present disclosure;

FIG. 3 schematically illustrates a flowchart of a part of blocks of executing a method for weight management benefit prediction by an electronic device according to still another example arrangement of the present disclosure;

FIG. 4 schematically illustrates a constitution block diagram of an apparatus for weight management benefit prediction according to an example arrangement of the present disclosure;

FIG. 5 schematically illustrates a flow block diagram of executing a method for weight management benefit prediction by an electronic device in an application scenario according to an example arrangement of the present disclosure;

FIG. 6 schematically illustrates a flow block diagram of a method for weight management benefit prediction in another application scenario according to an example arrangement of the present disclosure;

FIG. 7 schematically illustrates a schematic diagram of a program product according to an example arrangement of the present disclosure; and

FIG. 8 schematically illustrates a schematic modular diagram of an electronic device according to an example arrangement of the present disclosure.

### DETAILED DESCRIPTION

The example arrangement will now be described more fully with reference to the accompanying drawings. However, the example arrangements can be implemented in a variety of forms and should not be construed as limited to the arrangements set forth herein. Rather, the arrangements are provided so that the present disclosure will be thorough and complete and will fully convey the concepts of example arrangements to those skilled in the art. The described features, structures, or characteristics may be combined in any suitable manner in one or more arrangements.

In addition, the accompanying drawings are merely example illustration of the present disclosure, and are not necessarily drawn to scale. The same reference numerals in the drawings denote the same or similar parts, and thus repeated description thereof will be omitted. Some block diagrams shown in the figures are functional entities and not necessarily to be corresponding to a physically or logically individual entities. These functional entities may be implemented in software form, or implemented in one or more hardware modules or integrated circuits, or implemented in different networks and/or processor apparatuses and/or microcontroller apparatuses.

An example arrangement of the present disclosure first provides a method for operating an electronic device to perform a method for weight management benefit prediction. This method may be used to make a prediction of risk of suffering a target disease induced by a plurality of factors such as overweight or obesity, and to make quantitative prediction of weight management benefits based on prediction results.

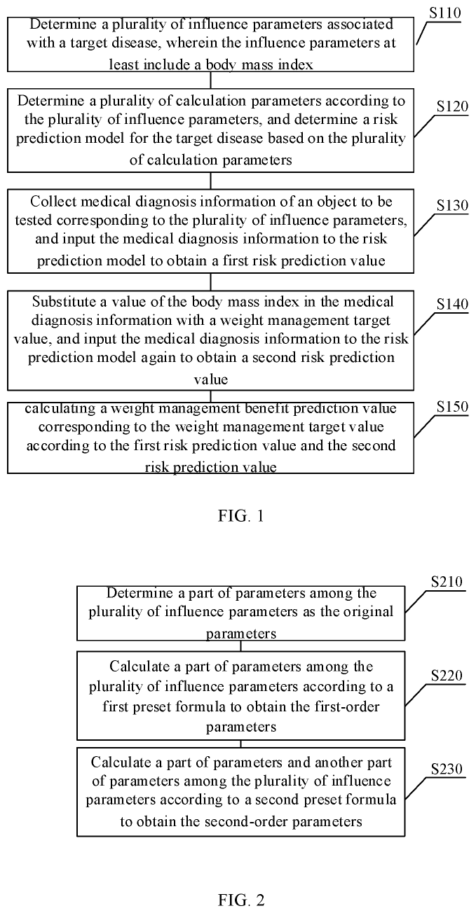

As shown in FIG. 1, the method for weight management benefit prediction executed by the electronic device according to this example arrangement may mainly include following blocks.

Block S110: determining a plurality of influence parameters associated with a target disease. The influence parameters at least include a body mass index.

A plurality of influence parameters associated with a target disease to be tested is first determined in this block. The target disease may be a type of disease in which the risk of suffering from the disease is highly associated with the weight of the tested population, and the influence parameters mainly include the body mass index and other factors that may influence the risk of suffering from the target disease. For example, if the target disease is auricular fibrillation, the influence parameters may include age, gender, body mass index (BMI), systolic pressure, treatment of hypertension, PR interval, abnormal heart sounds, and history of heart failure. For another example, if the target disease is cardiovascular disease, the influence parameters may include age, gender, BMI, systolic pressure, treatment of hypertension, smoking status, and diabetes. In this example arrangement, a mapping relationship between various types of diseases and influence factors may be established based on statistical data, and a plurality of influence parameters associated with the target disease may be determined by querying the corresponding mapping relationship table.

Block S120: determining a plurality of calculation parameters according to the plurality of influence parameters, and determining a risk prediction model for the target disease based on the plurality of calculation parameters.

A plurality of calculation parameters may be determined in this block according to the plurality of influence parameters obtained in Block S110, and then a risk prediction model is determined for the target disease based on the calculation parameters. The calculation parameters are obtained after a certain operation processing is performed on the influence parameters. Manners for determining the calculation parameters may be different for different target diseases and different influence parameters. For example, for a certain target disease, the influence parameters include age, BMI, PR interval, etc. The calculation parameters determined according to the influence parameters may include the square of the age, the product of the age and the BMI, the product of the BMI and the PR interval, and the like. The risk prediction model is a calculation model taking the determined calculation parameters as input variables, and its output result is a risk of suffering from the target disease. In this example arrangement, a plurality of prediction model templates may be preset, and then the risk prediction model for the target disease is formed according to the determined calculation parameters.

Block S130: collecting medical diagnosis information of an object to be tested corresponding to the plurality of influence parameters, and inputting the medical diagnosis information to the risk prediction model to obtain a first risk prediction value.

In this block, the medical diagnosis information of the object to be tested may be specifically collected according to the influence parameters determined in Block S110, and the medical diagnosis information collected is inputted to the risk prediction model determined in Block S120 to obtain the first risk prediction value. The first risk prediction value is a value predicted for the risk of suffering from the target disease based on the medical diagnosis information of the object to be tested.

Block S140: substituting a value of the body mass index in the medical diagnosis information with a weight management target value, and inputting the medical diagnosis information to the risk prediction model again to obtain a second risk prediction value.

To predict the weight management benefits of the object to be tested in terms of the risk of suffering from the target disease, a weight management target value will be preset in this block, and then the body mass index in the collected medical diagnosis information of the object to be tested is substituted with the weight management target value, and then the substituted medical diagnosis information is inputted to the risk prediction model again to obtain the second risk prediction value. The same prediction model and the same calculation parameters are used in the calculation of the first risk prediction value and the second risk prediction value. The difference therebetween lies in that one of the influence parameters used to calculate the first risk prediction value is the actually-collected body mass index of the object to be tested, whereas the corresponding influence parameter used to calculate the second risk prediction value is a preset weight management target value. For example, the body mass index of the object to be tested is 26.3, whereas the weight management target value may be 25.

Block S150: calculating according to the first risk prediction value and the second risk prediction value to obtain a weight management benefit prediction value corresponding to the weight management target value.

After respectively calculating and obtaining the first risk prediction value and the second risk prediction value of the object to be tested for the target disease, the weight management benefit prediction value corresponding to the weight management target value may be obtained in this block according to risk prediction results. The method of calculating the weight management benefit prediction value may be to subtract the first risk prediction value from the second risk prediction value to obtain a weight management benefit prediction value corresponding to the weight management target value. The weight management income prediction value reflects effective benefits that may be obtained in terms of the risk of suffering from the target disease based on the currently collected medical diagnosis information of the object to be tested when the weight indication is adjusted to be the weight management target value by various weight management control methods such as exercise fitness, reasonable diet, and medical treatment, etc. Moreover, as quantized data, the weight management benefit prediction value may intuitively allow the object to be tested to feel the effect of the weight management benefits.

The electronic device provided by this example arrangement may be used for executing the method for weight management benefit prediction. Quantitative assessment of benefits produced by effective weight management may be implemented based on calculation results of the risk prediction model, quantitative benefit measurement results may be provided from the perspective of health risk assessment, making it easy for non-medical professionals to understand and accept significance of weight management, and thus promoting prevention and control of overweight or obesity, a common health risk factor.

The health risk assessment is used to describe and assess the possibility of an individual's future occurrence of a particular disease or death due to a specific disease. Generally, a questionnaire is used to collect information of the person to be assessed, and an internal algorithm is used to predict the disease risk of the person to be assessed. The method of assessing the disease risk is directly derived from epidemiological research results. Prospective cohort studies and comprehensive analysis of past epidemiological research results and evidence-based medicine are the leading methods. Framingham Heart Study is a long-term prospective cohort in the field of cardiovascular diseases, and in the example arrangements of the present disclosure, a risk prediction model for the cardiovascular disease may be determined based on the Framingham Heart Study.

For example, in an example arrangement of the present disclosure, the risk prediction model may be selected as below: Z=1−a\hat{ }(sum(βᵢ*Xᵢ)−b). Z represents a risk prediction value of the target disease, Xᵢ represents the plurality of calculation parameters, βᵢ represents a preset weight coefficient of the plurality of calculation parameters, and a and b represent preset regulation coefficients.

For another example, in another example arrangement of the present disclosure, the risk prediction model may be selected as below: Z=1−c\hat{ }(sum(βᵢ*(Yᵢ−Xᵢ))).

Z represents a risk prediction value of the target disease, Xᵢ represents the plurality of calculation parameters, βᵢ represents a preset weight coefficient of the plurality of calculation parameters, c represents a preset regulation coefficient, and Yᵢ represents preset reference values of the plurality of calculation parameters.

On the basis of the above example arrangement, the influence parameters determined in Block S110 may include numerical parameters and non-numerical parameters. Correspondingly, before determining a plurality of calculation parameters according to the plurality of influence parameters in Block S120, the method for weight management benefit prediction may further include: converting the non-numerical parameters in the influence parameters into the numerical parameters. One of the conversion methods may be to establish a mapping relationship between a specific value of the non-numeric parameter and a preset value. For example, for the influence parameter “gender”, “male” may be mapped to 1 and “female” may be mapped to 0. For the influence parameter “treatment of hypertension”, “treated” may be mapped to 1, and “untreated” may be mapped to 0. In some other example arrangements, any other numerical conversion methods may also be employed, which is not specifically limited in the present disclosure. By way of numerical conversion, the range of collection of the influence parameters may be expanded, and the prediction dimension of the risk prediction model may be improved.

As shown in FIG. 2, in another example arrangement of the present disclosure, the calculation parameters determined in Block S120 may further include original parameters, first-order parameters, and second-order parameters. Correspondingly, the determining a plurality of calculation parameters according to the plurality of influence parameters may further include following blocks.

Block S210: determining a part of parameters among the plurality of influence parameters as the original parameters.

A part of parameters among the influence parameters may be directly used as the original parameters, such as gender, age, BMI, systolic pressure, and so on.

Block S220: calculating a part of parameters among the plurality of influence parameters according to a first preset formula to obtain the first-order parameters.

The first-order parameters may be obtained by calculating another part of parameters among the influence parameters according to the first preset formula, for example, squaring the age, or taking a natural logarithm to the systolic pressure, etc.

Block S230: calculating a part of parameters and another part of parameters among the plurality of influence parameters according to a second preset formula to obtain the second-order parameters.

In this block, a part of parameters and another part of parameters among the plurality of influence factors may be jointly calculated according to the second preset formula to obtain the second-order parameters, for example, multiplying the age with the “treatment of hypertension”, and for another example, multiplying the gender with the square of the age.

In this example arrangement, various forms of calculation parameters may be obtained by integrating the influence parameters, and the assessment analysis dimension of the risk prediction model may be increased.

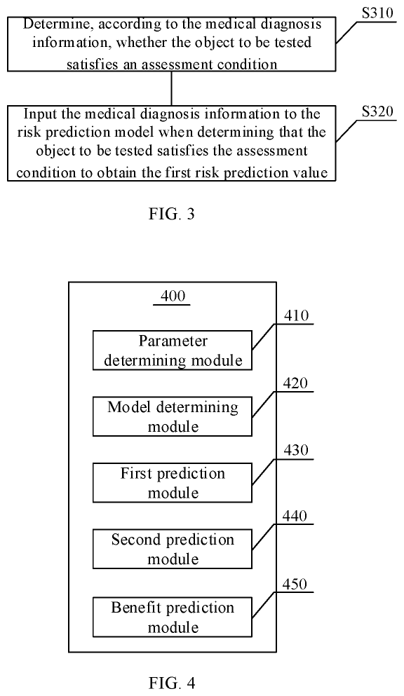

As shown in FIG. 3, in another example arrangement of the present disclosure, the Block S130 of inputting the medical diagnosis information to the risk prediction model to obtain a first risk prediction value may further include following blocks.

Block S310: determining, according to the medical diagnosis information, whether the object to be tested satisfies an assessment condition.

In this block, first it may be determined, according to the collected medical diagnosis information, whether the object to be tested satisfies a preset assessment condition. The preset assessment condition may be, for example, the body mass index of the object to be tested is greater than a preset threshold, or may be, for another example, the age of the object to be tested is within a preset range.

Block S320: inputting the medical diagnosis information to the risk prediction model when determining that the object to be tested satisfies the assessment condition to obtain the first risk prediction value.

When determining that the object to be tested satisfies the preset assessment condition, the medical diagnosis information is inputted to the risk prediction model according to the determination results in Block S310 to obtain the first risk prediction value. If the determination results indicate that the object to be tested does not satisfy the preset assessment condition, a prompt message may be directly fed back, and a user is prompted that this risk prediction model cannot be applied to the current user to be tested.

In this example arrangement, users to be tested may be screened according to preset assessment conditions, such that pertinence and effectiveness of the risk prediction model may be improved.

It is to be noted that, blocks of the method in the present disclosure are described in a particular order in the above example arrangements. However, this does not require or imply to execute these blocks necessarily according to the particular order, or this does not mean that the expected result cannot be implemented unless all the blocks are executed. Additionally or alternatively, some blocks may be omitted, a plurality of blocks may be combined into one block for execution, and/or one block may be decomposed into a plurality of blocks for execution.

In an example arrangement of the present disclosure, there is also provided an apparatus for weight management benefit prediction, which corresponds to the method for weight management benefit prediction executed by the electronic device in the above arrangements. As shown in FIG. 4, the apparatus 400 for weight management benefit prediction may include: a parameter determining module 410, a model determining module 420, a first prediction module 430, a second prediction module 440, and a benefit prediction module 450.

The parameter determining module 410 is configured to determine a plurality of influence parameters associated with a target disease. The influence parameters at least include a body mass index. The parameter determining module 410 first determines a plurality of influence parameters associated with a target disease to be tested. The target disease may be a type of disease in which the risk of suffering from the disease is highly associated with the weight of the tested population, and the influence parameters mainly include the body mass index and other factors that may influence the risk of suffering from the target disease.

The model determining module 420 is configured to determine a plurality of calculation parameters according to the plurality of influence parameters, and determine a risk prediction model for the target disease based on the plurality of calculation parameters. The model determining module 420 may determine a plurality of calculation parameters according to the plurality of influence parameters obtained by the parameter determining module 410, and then may determine the risk prediction model for the target disease based on the plurality of calculation parameters. The calculation parameters are obtained after a certain operation processing is performed on the influence parameters. Manners for determining the calculation parameters may be different for different target diseases and different influence parameters. The risk prediction model is a calculation model taking the determined calculation parameters as input variables, and its output result is a risk of suffering from the target disease. In this example arrangement, a plurality of prediction model templates may be preset, and then the risk prediction model for the target disease is formed according to the determined calculation parameters.

The first prediction module 430 is configured to collect medical diagnosis information of an object to be tested corresponding to the plurality of influence parameters, and input the medical diagnosis information to the risk prediction model to obtain a first risk prediction value. The first prediction module 430 may collect medical diagnosis information of an object to be tested according to the influence parameters determined by the parameter determining module 410, and input the collected medical diagnosis information to the risk prediction model determined by the model determining module 420 to obtain a first risk prediction value. The first risk prediction value is a value predicted for the risk of suffering from the target disease based on the medical diagnosis information of the object to be tested.

The second prediction module 440 is configured to substitute a value of the body mass index in the medical diagnosis information with a weight management target value, and input the medical diagnosis information to the risk prediction model again to obtain a second risk prediction value. To predict the weight management benefits of the object to be tested in terms of the risk of suffering from the target disease, the second prediction module 440 will preset a weight management target value, and then substitute the value of the body mass index in the collected medical diagnosis information of the object to be tested with the weight management target value, and then input the substituted medical diagnosis information to the risk prediction model again to obtain the second risk prediction value. The same prediction model and the same calculation parameters are used in the calculation of the first risk prediction value and the second risk prediction value. The difference therebetween lies in that one of the influence parameters used to calculate the first risk prediction value is the actually-collected body mass index of the object to be tested, whereas the corresponding influence parameter used to calculate the second risk prediction value is a preset weight management target value.

The benefit prediction module 450 is configured to calculate and obtain a weight management benefit prediction value corresponding to the weight management target value according to the first risk prediction value and the second risk prediction value. After respectively calculating and obtaining the first risk prediction value and the second risk prediction value of the object to be tested for the target disease, benefit prediction module 450 may calculate and obtain the weight management benefit prediction value corresponding to the weight management target value according to risk prediction results. The weight management income prediction value reflects effective benefits that may be obtained in terms of the risk of suffering from the target disease based on the currently collected medical diagnosis information of the object to be tested when the weight indication is adjusted to be the weight management target value by various weight management control methods such as exercise fitness, reasonable diet, and medical treatment, etc. Moreover, as quantized data, the weight management benefit prediction value may intuitively allow the object to be tested to feel the effect of the weight management benefits.

In another example arrangement of the present disclosure, the calculation parameters determined by the model determining module 420 may further include original parameters, first-order parameters, and second-order parameters. Correspondingly, the model determining module 420 at least may further include an original parameter determining module, a one-dimensional parameter determining module, and a two-dimensional parameter determining module.

The original parameter determining module is configured to determine a part of parameters among the plurality of influence parameters as the original parameters. A part of parameters among the influence parameters may be directly used as the original parameters, such as gender, age, BMI, systolic pressure, and so on.

The one-dimensional parameter determining module is configured to calculate a part of parameters among the plurality of influence parameters according to a first preset formula to obtain the first-order parameters. The first-order parameters may be obtained by calculating another part of parameters among the influence parameters according to the first preset formula, for example, squaring the age, or taking a natural logarithm to the systolic pressure, etc.

The two-dimensional parameter determining module is configured to jointly calculate a part of parameters and another part of parameters among the plurality of influence parameters according to a second preset formula to obtain the second-order parameters. The two-dimensional parameter determining module may jointly calculate a part of parameters and another part of parameters among the plurality of influence parameters according to the second preset formula to obtain the second-order parameters. For example, multiplying the age with the “treatment of hypertension”, and for another example, multiplying the gender with the square of the age.

In another example arrangement of the present disclosure, the first prediction module 430 may further include an assessment module and a prediction module.

The assessment module is configured to determine, according to the medical diagnosis information, whether the object to be tested satisfies an assessment condition. The assessment module may first determine, according to the collected medical diagnosis information, whether the object to be tested satisfies a preset assessment condition. The preset assessment condition may be, for example, the body mass index of the object to be tested is greater than a preset threshold, or may be, for another example, the age of the object to be tested is within a preset range.

The prediction module is configured to input the medical diagnosis information to the risk prediction model when determining that the object to be tested satisfies the assessment condition to obtain the first risk prediction value. When determining that the object to be tested satisfies the preset assessment condition, the prediction module inputs the medical diagnosis information to the risk prediction model again according to the determination results of the assessment module to obtain the first risk prediction value. If the determination results indicate that the object to be tested does not satisfy the preset assessment condition, a prompt message may be directly fed back, and a user is prompted that this risk prediction model cannot be applied to the current user to be tested.

Specific details of the above apparatus for weight management benefit prediction have been described in detail in the corresponding method for weight management benefit prediction executed by the electronic device, and thus are not described in detail herein.

The parameter determining module 410, the model determining module 420, the first prediction module 430, the second prediction module 440, the benefit prediction module 450, the assessment module and the prediction module an original parameter determining module, the one-dimensional parameter determining module and the two-dimensional parameter determining module described above may be program unit that can be executed by the processor, or a chip capable of implementing the above operation blocks.

It is to be noticed that although a plurality of modules or units of the device for action execution have been mentioned in the above detailed description, this partition is not compulsory. Actually, according to the arrangement of the present disclosure, features and functions of two or more modules or units as described above may be embodied in one module or unit. Reversely, features and functions of one module or unit as described above may be further embodied in more modules or units.

The method for weight management benefit prediction and the apparatus for weight management benefit prediction provided by the above example arrangements are described in detail below with reference to specific application scenarios.

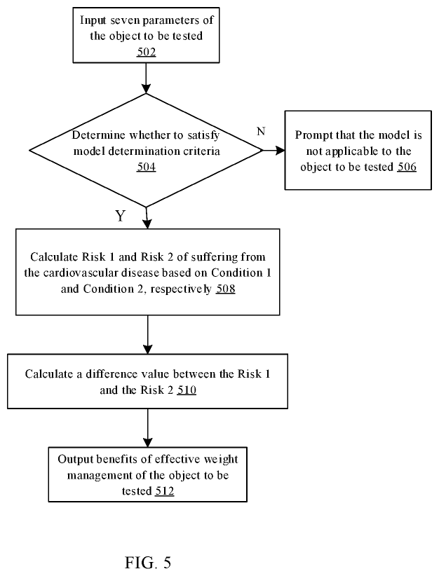

According to the present disclosure, in an application scenario, the cardiovascular disease may be taken as the target disease to make quantitative prediction of weight management benefits thereof. As shown in FIG. 5, specific prediction procedures include the following blocks.

i. Input Block 502

Seven indicators are inputted, i.e., the age, the gender, the BMI, the systolic pressure, the treatment of hypertension, the smoking status and diabetes of the object to be tested.

ii. Determining Block 504

This part of main functions is to determine whether people to be assessed are people to which the calculation model is applicable. Determination criteria are as follows:

(1) Middle-aged and elderly people aged 30 to 74 years old without cardiovascular diseases (except hypertension) (determined by the model).

(2) BMI>25 kg/m² (depending on demands).

If any one of the user's age and BMI does not satisfy the above criteria, it is prompted that this model is not applicable to the person to be assessed (block 506). Otherwise, a calculation block is entered (block 508).

iii. Calculation Block 508

After the basic determination, the risks of suffering from the cardiovascular disease are respectively calculated by adopting the following formula based on Condition 1 where the BMI=an input value and the other six indicators are input values and Condition 2 where BMI=25 and the other six indicators are input values, and a difference value between the disease risks in the two cases is calculated (block 510).

Benefits of effective weight management (in terms of cardiovascular disease)

=the reduction value (R %) of the risk of suffering from the cardiovascular disease for the first time within 10 years

### =Risk 2−Risk 1

The calculation of the risk is based on the results of the Framingham Heart Study, which is in detail as below:

for male, the risk=1−0.88431\hat{ }(sum(pX)−23.9388)); and

for female, the risk=1−0.94833\hat{ }(sum(pX)−26.0145)).

Values of the preset weight coefficient β corresponding to each calculation parameter are seen in the following table. X represents the input value of the calculation parameter.

| Indicator (unit) | Male β | Female β |
|---|---|---|
| Natural logarithm of age (years old) | 3.11296 | 2.72107 |
| BMI (kg/m²) | 0.79277 | 0.51125 |
| Treatment of hypertension (determination criteria) | | |
| Natural logarithm of systolic pressure (mmHg) | 1.85508 | 2.88267 |
| treated | | |
| Natural logarithm of systolic pressure (mmHg) | 1.92672 | 2.81291 |
| untreated | | |
| Smoking (Y 1, N 0) | 0.70953 | 0.61868 |
| Diabetes (Y 1, N 0) | 0.53160 | 0.77763 |

iv. Output Block 512

If the user's age or BMI does not meet the relevant determination criteria, it is prompted that the model is not applicable to the person to be assessed.

If the relevant determination criteria are met, the benefits of effective weight management are outputted, it is prompted that the risk of suffering from the cardiovascular disease within 10 years can be minimized by R % if the weight can be managed within the normal range within 1 year, and weight management suggestions are provided in terms of diet, exercise, and medical treatment, etc.

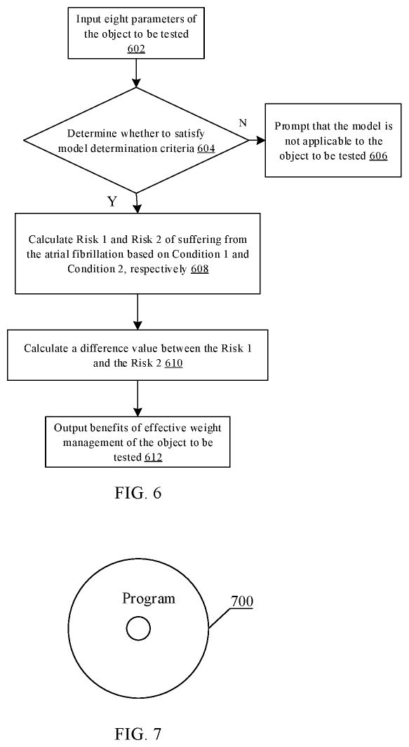

According to the present disclosure, in another application scenario, the auricular fibrillation (i.e., atrial fibrillation) may be taken as the target disease to make quantitative prediction of weight management benefits thereof. As shown in FIG. 6, specific prediction procedures include the following blocks.

i. Input Block 602

Eight indicators are inputted, i.e., the age, the gender, the BMI, the systolic pressure, the treatment of hypertension, the PR interval, the abnormal heart sounds, and the history of heart failure of the object to be tested.

ii. Determining Block 604

This part of main functions is to determine whether people to be assessed are people to which the calculation model is applicable. Determination criteria are as follows:

(1) Middle-aged and elderly people aged 45 to 95 years old without atrial fibrillation (determined by the model).

(2) BMI>25 kg/m² (depending on demands).

If any one of the user's age and BMI does not satisfy the above criteria, it is prompted that this model is not applicable to the person to be assessed (block 606). Otherwise, a calculation block is entered (block 608).

iii. Calculation Block 608

After the basic determination, the risks of suffering from the atrial fibrillation are respectively calculated by adopting the following formula based on Condition 1 where the BMI=an input value and the other seven indicators are input values and Condition 2 where BMI=25 and the other seven indicators are input values, and a difference value between the disease risks in the two cases is calculated (block 610).

Benefits of effective weight management (in terms of atrial fibrillation)

=the reduction value (R %) of the risk of suffering from the atrial fibrillation for the first time within 10 years

=Risk 2−Risk 1

The calculation of the risk is based on the results of the Framingham Heart Study, which is in detail as below:

The risk=1−0.96337\hat{ }(sum(βᵢ*(Yᵢ−Xᵢ)))

Values of the preset weight coefficient β corresponding to each calculation parameter and the preset reference value Y are seen in the following table. X represents the input value of the calculation parameter.

| Indicator (unit) | β | Y |
|---|---|---|
| Gender (male 1, female 0) | 1.994060 | 0.4464 |
| Age (years old) | 0.150520 | 60.9022 |
| BMI (kg/m²) | 0.019300 | 26.2861 |
| Systolic pressure (mmHg) | 0.006150 | 136.1674 |
| Treatment of hypertension (treated 1, untreated 0) | 0.424100 | 0.2413 |
| PR interval (ms) | 0.070650 | 16.3901 |
| Abnormal heart sounds (Y 1, N 0) | 3.795860 | 0.0281 |
| History of heart failure (Y 1, N 0) | 9.428330 | 0.0087 |
| Square of age | −0.000380 | 3806.9000 |
| Gender multiplied by square of age | −0.000280 | 1654.6600 |
| Age multiplied by treatment of hypertension | −0.042380 | 1.8961 |
| Age multiplied by history of heart failure | −0.123070 | 0.6100 |

iv. Output Block 612

If the user's age or BMI does not meet the determination criteria, it is prompted that the model is not applicable to the person to be assessed.

If the determination criteria are met, the benefits of effective weight management are outputted, it is prompted that the risk of suffering from the atrial fibrillation within 10 years can be minimized by R % if the weight can be managed within the normal range within 1 year, and weight management suggestions are provided in terms of diet, exercise, and medical treatment, etc.

In an example arrangement of the present disclosure, there is also provided a computer-readable storage medium, which stores a computer program. The computer program is executable by a processor, whereby the above method for weight management benefit prediction of the present disclosure may be implemented. In some possible arrangements, aspects of the present disclosure may be implemented as a form of a program product, which includes a program code. The program product may be stored in a nonvolatile storage medium (which may be CD-ROM, USB flash disk, or mobile hard disk and the like) or on network. When the program product runs on a computing device (which may be a personal computer, a server, a terminal device, or a network device and the like), the program code is used for enabling the computing device to perform the blocks of the method described in the above example arrangements of the present disclosure.

Referring to FIG. 7, a program product 700 configured to implement the above method is described according to the arrangements of the present disclosure. The program product 700 may adopt a portable compact disc read-only memory (CD-ROM) and include a program code, and may run on a computing device (such as a personal computer, a server, a terminal device, or a network device and the like). However, the program product of the present disclosure is not limited thereto. In this example arrangement, the computer-readable storage medium may be any tangible medium that can contain or store a program for use by or in connection with an instruction execution system, apparatus, or device.

Any combination of one or more readable medium(s) may be utilized by the program product. The readable medium may be a readable signal medium or a readable storage medium.

The readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the readable storage medium include the following: an electrical connection having one or more wires, a portable diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing.

The readable signal medium may include a propagated data signal with readable program code embodied therein, for example, in baseband or as part of a carrier wave. Such a propagated data signal may take any of a variety of forms, including, but not limited to, electro-magnetic, optical, or any suitable combination thereof. The readable signal medium may be any readable medium that is not a readable storage medium and that can transmit, propagate, or transport a program for use by or in connection with an instruction execution system, apparatus, or device.

Program code embodied on the readable medium may be transmitted using any appropriate medium, including but not limited to wireless, wireline, optical fiber cable, RF, etc., or any suitable combination of the foregoing.

Program code for carrying out operations of the present disclosure may be written in any combination of one or more programming languages, including an object-oriented programming language such as Java, C++ or the like and conventional procedural programming languages, such as the “C” programming language or similar programming languages. The program code may execute entirely on the user's computing device, partly on the user's computing device, as a stand-alone software package, partly on the user's computing device and partly on a remote computing device or entirely on the remote computing device or server. In the latter scenario, the remote computing device may be coupled to the user's computing device through any type of network, including a local area network (LAN) or a wide area network (WAN), or may be coupled to an external computing device (for example, through the Internet using an Internet Service Provider).

In an example arrangement of the present disclosure, there is also provided an electronic device, which includes at least one processor and at least one memory configured to store executable instructions of the processor. The processor is configured to perform blocks of the method in the above example arrangements of the present disclosure by executing the executable instructions.

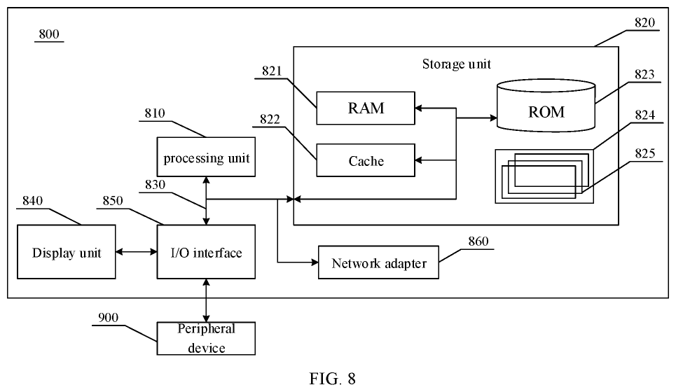

The electronic device 800 in this example arrangement is described below with reference to FIG. 8. The electronic device 800 is merely an example, and no limitation should be imposed on functions or scope of use of the arrangements of the present disclosure.

As shown in FIG. 8, the electronic device 800 is shown in the form of a general-purpose computing device. Components of the electronic device 800 may include, but are not limited to: at least one processing unit 810, at least one storage unit 820, a bus 830 connecting different system components (including the processing unit 810 and the storage unit 820), and a display unit 840.

The storage unit 820 stores a program code, which may be executed by the processing unit 810, such that the processing unit 810 performs blocks of the method described in the example arrangements of the present disclosure.

The storage unit 820 may include readable media in the form of volatile storage unit, such as a random access memory (RAM) 821 and/or a cache memory 822. Furthermore, the storage unit 820 may further include a read-only memory (ROM) 823.

The storage unit 820 may further include a program/utility tool 824 having a group of (at least one) program modules 825. The program modules 825 include, but are not limited to: an operating system, one or more applications, other program modules and program data. Each or a certain combination of these examples may include implementation of network environment.

The bus 830 may represent one or more of a plurality of bus structures, including a memory bus or memory controller, a peripheral bus, an accelerated graphics port, a processing unit or a local bus using any bus structure among the plurality of bus structures.

The electronic device 800 may communicate with one or more peripheral devices 900 (such as keyboards, pointing devices, Bluetooth devices, etc.), and also may communicate with one or more devices allowing a user to interact with the electronic device 800, and/or may communicate with any device (for example, a router, a modem and so on) allowing the electronic device 800 to communicate with one or more other computing devices. This communication may be implemented by means of an input/output (I/O) interface 850. Moreover, the electronic device 800 also may communicate with one or more networks (for example, a local area network (LAN), a wide area network (WAN) and/or a public network such as the Internet) via a network adapter 860. As shown in FIG. 8, the network adapter 860 may communicate with other modules of the electronic device 800 through the bus 830. It should be understood that although not shown in the figures, other hardware and/or software modules may be used in combination with the electronic device 800, including but not limited to: microcode, device drivers, redundancy processing units, external disk drive arrays, redundant arrays of independent disks (RAID) systems, tape drives and data backup and storage systems, etc.

As will be appreciated by one skilled in the art, aspects of the present disclosure may be embodied as a system, method or program product. Accordingly, aspects of the present disclosure may take the form of an entirely hardware arrangement, an entirely software arrangement (including firmware, micro-code, etc.) or an arrangement combining software and hardware aspects that may all generally be referred to herein as a “circuit,” “module” or “system.”

Other arrangements of the present disclosure will be apparent to those skilled in the art from consideration of the specification and practice of the current disclosure. This application is intended to cover any variations, uses, or adaptations of the present disclosure following the general principles thereof and including such departures from the present disclosure as come within known or customary practice in the art. It is intended that the specification and arrangements be considered as example only, with a true scope and spirit of the present disclosure being indicated by the appended claims.

The features, structures, or characteristics described above may be combined in one or more arrangements in any suitable manner, and the features discussed in each arrangement are interchangeable, if possible. In the following description, numerous specific details are provided to provide a thorough understanding of the arrangements of the present disclosure. However, those skilled in the art will appreciate that the technical solutions in the present disclosure may be practiced without one or more of the specific details, or other methods, modules, materials and so on may be employed. In other circumstances, well-known structures, materials or operations are not shown or described in detail to avoid confusion of respective aspects of the present disclosure.

What is claimed is:

1. A method for operating an electronic device comprising:
determining a plurality of influence parameters associated with a target disease, the influence parameters at least comprising a body mass index;
determining a plurality of calculation parameters according to the plurality of influence parameters, and determining a risk prediction model for the target disease based on the plurality of calculation parameters;
collecting medical diagnosis information of an object to be tested corresponding to the plurality of influence parameters, and inputting the medical diagnosis information to the risk prediction model to obtain a first risk prediction value;
substituting a value of the body mass index in the medical diagnosis information with a weight management target value, and inputting the medical diagnosis information to the risk prediction model again to obtain a second risk prediction value; and
calculating and obtaining a weight management benefit prediction value corresponding to the weight management target value according to the first risk prediction value and the second risk prediction value.

2. The method for operating an electronic device according to claim 1, wherein the risk prediction model is:
Z=1−a\hat{ }(sum(βᵢ*Xᵢ)−b)
wherein Z represents a risk prediction value of the target disease, Xᵢ represents the plurality of calculation parameters, βᵢ represents a preset weight coefficient of the plurality of calculation parameters, and a and b represent preset regulation coefficients.

3. The method for operating an electronic device according to claim 1, wherein the risk prediction model is:
Z=1−c\hat{ }(sum(βᵢ*(Yᵢ−Xᵢ)))
wherein Z represents a risk prediction value of the target disease, Xᵢ represents the plurality of calculation parameters, βᵢ represents a preset weight coefficient of the plurality of calculation parameters, c represents a preset regulation coefficient, and Yᵢ represents preset reference values of the plurality of calculation parameters.

4. The method for operating an electronic device according to claim 1, wherein the influence parameters comprise numerical parameters and non-numerical parameters; and
before determining a plurality of calculation parameters according to the plurality of influence parameters, the method further comprises: converting the non-numerical parameters in the influence parameters into the numerical parameters.

5. The method for operating an electronic device according to claim 4, wherein the calculation parameters comprise original parameters, first-order parameters, and second-order parameters, and wherein
determining a plurality of calculation parameters according to the plurality of influence parameters comprises:
determining one or more first parameters among the plurality of influence parameters as the original parameters;
calculating one or more second parameters among the plurality of influence parameters according to a first preset formula to obtain the first-order parameters; and
calculating one or more third parameters and one or more fourth parameters among the plurality of influence parameters according to a second preset formula to obtain the second-order parameters.

6. The method for operating an electronic device according to claim 1, wherein inputting the medical diagnosis information to the risk prediction model to obtain a first risk prediction value comprises:
determining, according to the medical diagnosis information, whether the object to be tested satisfies an assessment condition; and
inputting the medical diagnosis information to the risk prediction model when determining that the object to be tested satisfies the assessment condition to obtain the first risk prediction value.

7. The method for operating an electronic device according to claim 6, wherein the assessment condition comprises: the body mass index of the object to be tested being greater than a preset threshold.

8. The method for operating an electronic device according to claim 1, wherein determining a plurality of influence parameters associated with a target disease comprises:
querying a preset mapping relationship table to determine the plurality of influence parameters associated with the target disease; wherein the mapping relationship table is used for providing a mapping relationship between various diseases and the influence parameters.

9. The method for operating an electronic device according to claim 2, wherein determining a plurality of influence parameters associated with a target disease comprises:
querying a preset mapping relationship table to determine the plurality of influence parameters associated with the target disease; wherein the mapping relationship table is used for providing a mapping relationship between various diseases and the influence parameters.

10. The method for operating an electronic device according to claim 3, wherein determining a plurality of influence parameters associated with a target disease comprises:
querying a preset mapping relationship table to determine the plurality of influence parameters associated with the target disease; wherein the mapping relationship table is used for providing a mapping relationship between various diseases and the influence parameters.

11. The method for operating an electronic device according to claim 4, wherein determining a plurality of influence parameters associated with a target disease comprises:
querying a preset mapping relationship table to determine the plurality of influence parameters associated with the target disease; wherein the mapping relationship table is used for providing a mapping relationship between various diseases and the influence parameters.

12. The method for operating an electronic device according to claim 5, wherein determining a plurality of influence parameters associated with a target disease comprises:
querying a preset mapping relationship table to determine the plurality of influence parameters associated with the target disease; wherein the mapping relationship table is used for providing a mapping relationship between various diseases and the influence parameters.

13. The method for operating an electronic device according to claim 6, wherein determining a plurality of influence parameters associated with a target disease comprises:
querying a preset mapping relationship table to determine the plurality of influence parameters associated with the target disease; wherein the mapping relationship table is used for providing a mapping relationship between various diseases and the influence parameters.

14. The method for operating an electronic device according to claim 7, wherein determining a plurality of influence parameters associated with a target disease comprises:
querying a preset mapping relationship table to determine the plurality of influence parameters associated with the target disease; wherein the mapping relationship table is used for providing a mapping relationship between various diseases and the influence parameters.

15. An apparatus for weight management benefit prediction, comprising:
a parameter determining module, configured to determine a plurality of influence parameters associated with a target disease, wherein the influence parameters at least comprise a body mass index;
a model determining module, configured to determine a plurality of calculation parameters according to the plurality of influence parameters, and determine a risk prediction model for the target disease based on the plurality of calculation parameters;
a first prediction module, configured to collect medical diagnosis information of an object to be tested corresponding to the plurality of influence parameters, and input the medical diagnosis information to the risk prediction model to obtain a first risk prediction value;
a second prediction module, configured to substitute a value of the body mass index in the medical diagnosis information with a weight management target value, and input the medical diagnosis information to the risk prediction model again to obtain a second risk prediction value; and
a benefit prediction module, configured to calculate according to the first risk prediction value and the second risk prediction value to obtain a weight management benefit prediction value corresponding to the weight management target value.

16. A computer-readable storage medium, storing a computer program, wherein the computer program is executable by the processor, whereby the apparatus is configured to: determine a plurality of influence parameters associated with a target disease, wherein the influence parameters at least comprise a body mass index;
determine a plurality of calculation parameters according to the plurality of influence parameters, and determine a risk prediction model for the target disease based on the plurality of calculation parameters;
collect medical diagnosis information of an object to be tested corresponding to the plurality of influence parameters, and input the medical diagnosis information to the risk prediction model to obtain a first risk prediction value;
substitute a value of the body mass index in the medical diagnosis information with a weight management target value, and input the medical diagnosis information to the risk prediction model again to obtain a second risk prediction value; and
calculate according to the first risk prediction value and the second risk prediction value to obtain a weight management benefit prediction value corresponding to the weight management target value.
